(19) United States
(12) Patent Application Publication
(10) Pub. No.: US 2014/0316200 A1
(43) Pub. Date: Oct. 23, 2014

(54) OPTICAL TROCAR ASSEMBLY AND SYSTEM

(71) Applicants: Vhairi Maxwell, Leeds (GB); Nicholas Robinson, Leeds (GB)

(72) Inventors: Vhairi Maxwell, Leeds (GB); Nicholas Robinson, Leeds (GB)

(73) Assignee: Surgivcal Innovations Limited, Leeds, West Yorkshire (GB)

(21) Appl. No.: 14/233,688

(22) PCT Filed: Jul. 4, 2012
(86) PCT No.: PCT/GB2012/051559

§ 371 Date: Jul. 8, 2014

(30) Foreign Application Priority Data
Jul. 19, 2011 (GB) 1112366.8

(51) Int. Cl. A61B 1/00

(52) U.S. Cl. CPC A61B 1/00154
USPC 600/114

(57) ABSTRACT

An optical trocar assembly has an elongated tubular member for receiving an endoscope which defines a longitudinal axis and has an open proximal end and a closed, optically clear distal end which is adapted for penetrating body tissue. The optical trocar assembly has a handle releasably connected to the elongated tubular member and which defines a passage which is coaxial with the longitudinal axis and which has a distal portion and proximal portion. The distal portion receives the open proximal end and the proximal portion defines a tapering opening. A locking collet is positioned within the tapering opening having a through bore which is coaxial with the longitudinal axis and the tapering external surface. One of the tapering opening and the tapering external surface has a helical rib and the other of the tapering opening and the tapering external surface defines a helical recess engaging the helical rib

The present application relates to an optical trocar assembly and in particular to an optical trocar assembly that can be locked relative to an endoscope located within the optical trocar assembly in use.

Optical trocars are known. They comprise an elongated shaft with a clear distal end. In use, an endoscope is inserted inside the shaft so that it provides images taken through the optically transparent distal end. The distal end may have a variety of configurations suitable for penetrating tissue, for example it may taper towards a point. In use, the optical trocar enables visualisation of the passage of the trocar through body tissue.

It is desirable to lock the endoscope within the optical trocar so that movement relative to the optical trocar is prevented. A variety of systems have been proposed.

US-2005/0065543A1 (Kahle et al) discusses a bladeless optical obturator. It provides an obturator shaft into which an optical instrument is inserted. A lock may be disposed at the proximal end of the shaft to frictionally lock the optical instrument in an axial position in the shaft. The lock operates to prevent the optical instrument from moving axially relative to the shaft while allowing the optical instrument to rotate freely about the shaft. The lock can be a multi-finger collet having an inner diameter smaller than an outer diameter of the optical instrument wherein the fingers of the collet spread open during insertion with the optical instrument providing frictional engagement with the outer diameter of the optical instrument. A locking collar may also be provided to rotationally lock the optical instrument.

EP-1712196A2 (Tyco Healthcare Group LP) relates to an optical trocar with a scope holding assembly. The scope holding assembly includes a locking collet coaxially mounted relative to a longitudinal axis of a shaft in which the endoscope is received. A manual member is mounted adjacent the locking collet. In use, the manual member is rotated about the longitudinal axis to reduce an internal dimension of the internal passage of the locking collet. The resulting construction enables axial locking of the endoscope while still enabling the endoscope to rotate within the shaft.

In both of the above referenced documents, collets are used to prevent axial movement of an inserted endoscope, but the endoscope is free to rotate relative to the shaft. It would be desirable to lock an inserted endoscope against rotation about the longitudinal axis as well as against axial movement with a single action.

An optical trocar assembly is typically assembled in the operating theatre. It would also be desirable to provide a simple construction with low part count to simplify assembly and disassembly.

According to the present invention there is provided an optical trocar assembly comprising:
an elongated tubular member for receiving an endoscope, defining a longitudinal axis and having an open proximal end and a closed, optically clear distal end, wherein the distal end is adapted for penetrating body tissue;
a handle releasably connected to the elongated tubular member and defining a passage which is coaxial with the longitudinal axis and which has a distal portion and proximal portion, wherein the distal portion is for receiving the open proximal end and proximal portion defines a tapering opening which is tapered in the direction towards the distal portion; and
a locking collet positioned within the tapering opening, having a through bore which is coaxial with the longitudinal axis and a tapering external surface;
wherein one of the tapering opening and the tapering external surface comprises a helical rib and the other of the tapering opening and the tapering external surface defines a helical recess for engaging the helical rib.

Throughout this document, “distal” is used to refer to the direction away from a user of the optical trocar assembly and “proximal” is used to refer to the direction towards a user of the optical trocar assembly.

The assembly of the invention comprises three main parts, giving a simple construction which is easy to assemble prior to use and disassemble after use. The locking collet is captivated directly in the handle, this reduces loose components that may be lost if the handle during sterilisation/decontamination. A further advantage is that the low part count reduces production cost. It may also make sterilisation and/or decontamination easier between uses if the assembly is reusable.

The locking collet used in the present invention has a through bore which enables the endoscope to pass through. In common with other collets, the locking collet of the invention typically has longitudinal slots extending parallel to the longitudinal axis from the distal end of the locking collet into the tapering distal surface. These define tapering flexible members which can be deflected inwards into the through bore, reducing the effective diameter of the through bore. As the collet is advanced longitudinally into the tapering opening of the handle, the flexible members will deform inwards to grip an inserted endoscope. The longitudinal advancement of the locking collet occurs following rotation of the locking collet by the engagement of the helical rib with the helical recess. This construction provides a simple and effective way to hold an inserted endoscope both axially and rotationally relative to the longitudinal axis. Unlike the systems of US-2005/0065543A1 and EP-1712196A2, the present invention uses a collet to provide both axial and rotational locking and the collet is engaged by features provided in the handle itself, rather than requiring additional parts, for example the manual member required in EP-1712196A2.

In some embodiments, the helical rib and helical recess may be provided by a thread defined by the tapering opening and a corresponding thread defined by the tapering external recess.

Preferably, the locking collet further comprises an integrally formed and radially extending rotation member for rotating the locking collet relative to the handle. This provides easy operation and allows a user to achieve increased leverage for rotation, which may be required to ensure a good frictional lock onto an inserted endoscope.

The handle may further comprise first and second circumferential stops for engaging the rotation member and limiting the rotational movement of the locking collet to the handle. This can provide further ease of operation. For example, the pitch of the helical rib or thread may be chosen such that a rotation of the locking collet through less than 360° is sufficient to reduce the internal diameter so that an inserted endoscope is gripped securely. In that case, providing circumferential stops to limit the rotation of the locking collet can ensure that sufficient rotation is available for a secure grip, but that the locking collet cannot be over-rotated to a portion where damage may be caused to an inserted endoscope or parts of the assembly may break. A further advantage of this construction is that the position of the rotation member relative to the first and second circumferential stops gives immediate visual indication of whether an inserted endoscope is locked or unlocked relative to the optical trocar assembly.

In one embodiment, the handle further comprises a latch member for releasably engaging a latch surface formed on the proximal end of the elongated tubular member. This enables secure and releasable attachment between the handle and the elongated tubular member.

Preferably, the latch member is movable in a direction perpendicular to the longitudinal axis between open and closed positions; and wherein in the open position the proximal end of the elongated tubular member can be inserted into or removed from the handle, and in the closed position the latch surface of an inserted proximal end of the elongated tubular member is engaged by the latch member. The latch member may be biased into the closed position by a resilient element. Any suitable resilient element may be used, for example an elastomeric member or a spring, such as a helical spring.

Optionally, a portion of the latch member is accessible outside the handle to move the latch from the closed to the open position, against the force exerted by the resilient element. This allows a simple construction to allow access to the latch member for release of the latch where the disassembly is required. Alternatively, a latch release member may be provided which acts on the latch member against the force exerted by the resilient element to move the latch from the closed to the open position. However, this is less preferred because it increases the complexity of construction.

In another embodiment, the proximal end of the elongated tubular member further comprises a latch member having a projection which extends outwardly from the longitudinal axis; wherein the latch member is movable between a first position, in which the projection is at a first distance from the longitudinal axis and a second position, in which the projection is at a second distance from the longitudinal axis which is closer than the first distance; and wherein the handle further defines a receptacle which is engaged by the projection when the latch member is in the first position and disengaged by the projection when the latch member is in the second position.

In this embodiment, the latch member is provided on the elongated tubular member, rather than the housing. The construction of the housing is simplified because it only needs to define a receptacle to engage the projection of the latch member.

Preferably, the latch member is biased into the first position. This biasing may be achieved by a resilient element, such as an elastomer or a spring. It is preferred that the latch member is integrally formed with the elongated tubular member. In that case, the biasing into the first position can be achieved by using a resilient material for the elongated tubular member. When a resilient material is used for an integrally formed latch member, the latch member may be formed in the first position, so that it can be bent against the resilience of the material into the second position.

The latch member may further comprise an end portion positioned closer to the longitudinal axis than the projection; wherein when the latch member is in the first position, an internal cavity of the elongated tubular member is not obstructed by the end portion and, when the latch member is in the second position at least part of the end portion extends into the internal cavity. With this construction, accidental release of the latch when an endoscope is inserted is prevented. When an endoscope is inserted, movement of the end portion into the internal cavity of the elongated tubular member is prevented because it is filled by the endoscope. Therefore, movement of the latch member into the second position is prevented and the projection cannot be released from the receptacle.

Preferably, the end portion defines a concave curved surface. The concave curved surface preferably has a constant radius of curvature centred on the longitudinal axis. The radius of curvature may be chosen to correspond to the outer radius of an inserted endoscope.

At least two latch members may be provided in alternative embodiments. In that case the latch members are spaced around the longitudinal axis of the elongated tubular member at the same axial position. The at least two latch members may be evenly spaced or otherwise distributed around the longitudinal axis.

Preferably, the handle further comprises a grip extending substantially perpendicular to the longitudinal axis. This enables easier manipulation of the instrument by a user.

In another aspect of the invention, there is provided a system comprising an optical trocar as described above, with or without the optional features also described, and an endoscope sized to fit within the elongated tubular member, the passage of the handle and the through bore of the locking collet.

Embodiments of the invention will now be described by way of example, with reference to the accompanying drawings in which:

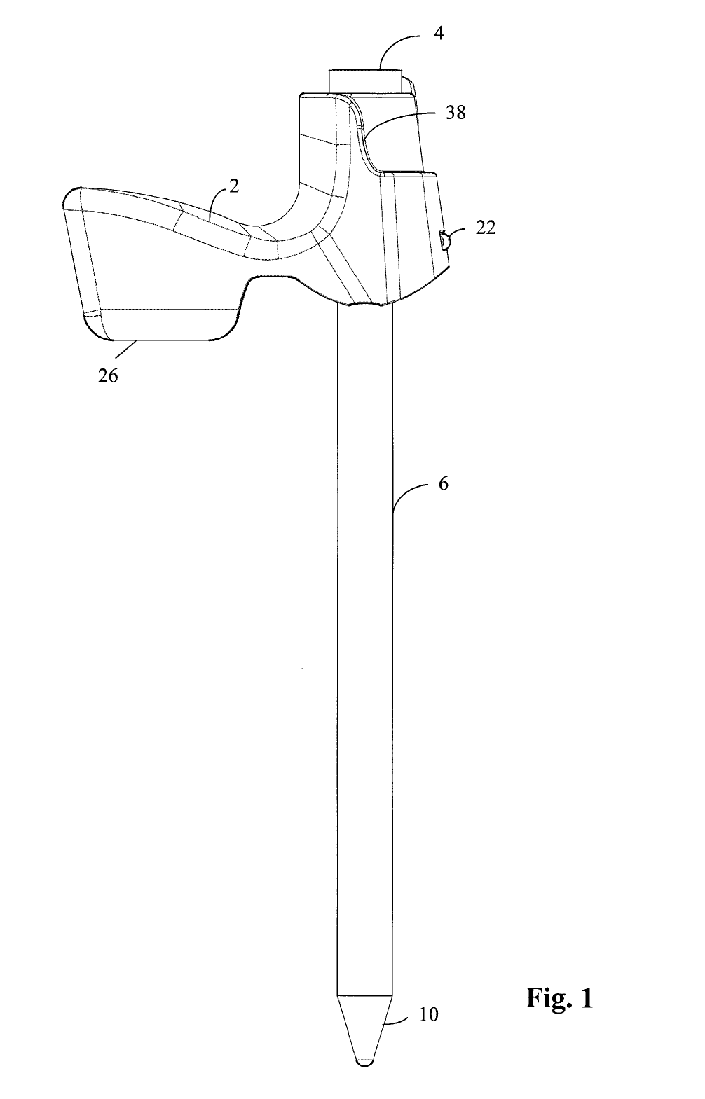

FIG. 1 is a side view of an optical trocar assembly according to a first embodiment of the present invention;

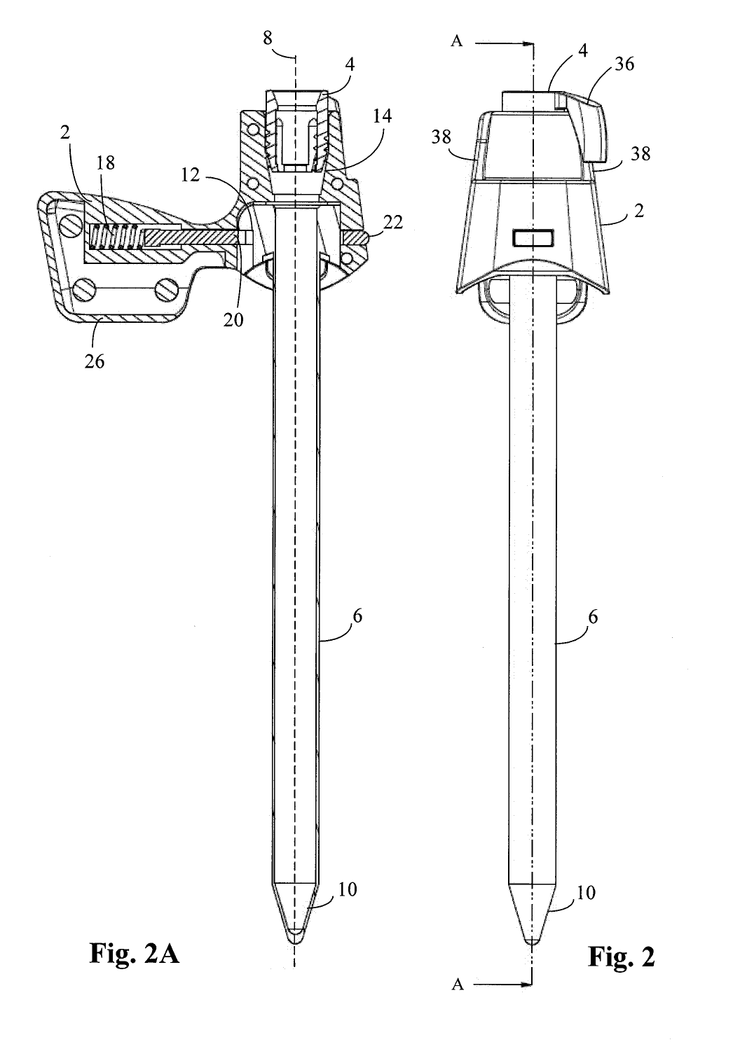

FIG. 2 is a top view of the optical trocar assembly of FIG. 1;

FIG. 2a is a cross-section through the optical trocar assembly of FIG. 1, taken along line A-A in FIG. 2;

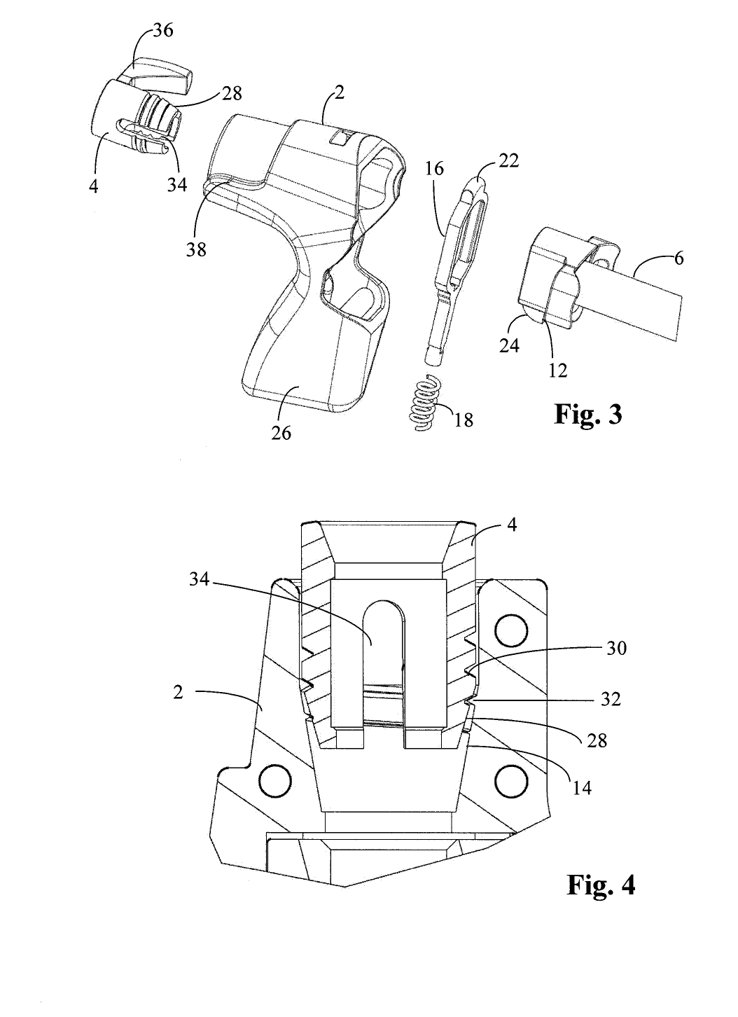

FIG. 3 is a partial exploded diagram showing the components of the optical trocar assembly of FIG. 1;

FIG. 4 is a close-up cross-section showing the engagement of the locking collet used in the embodiment of FIG. 1 with the tapering opening in the handle;

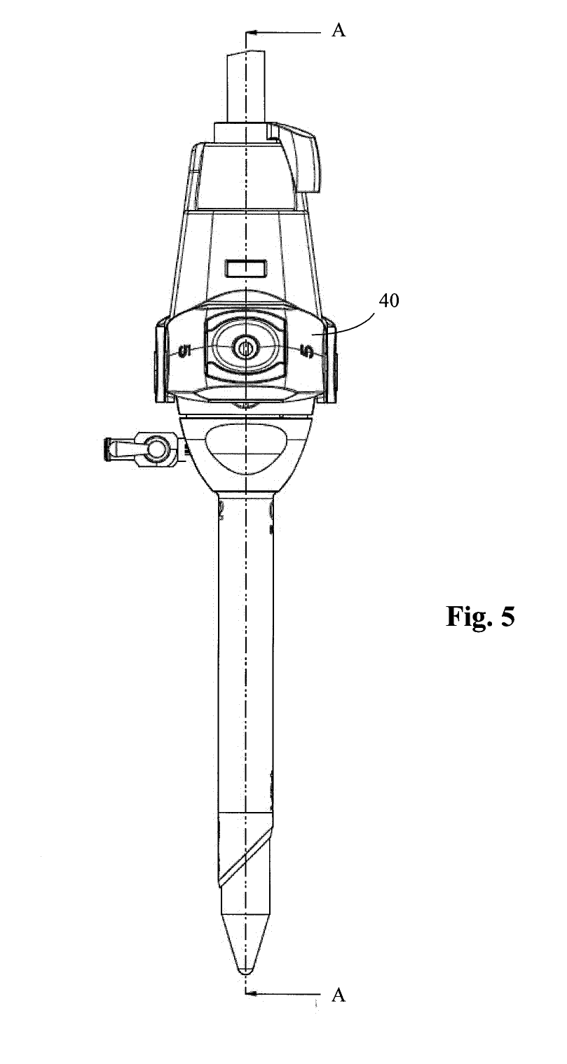

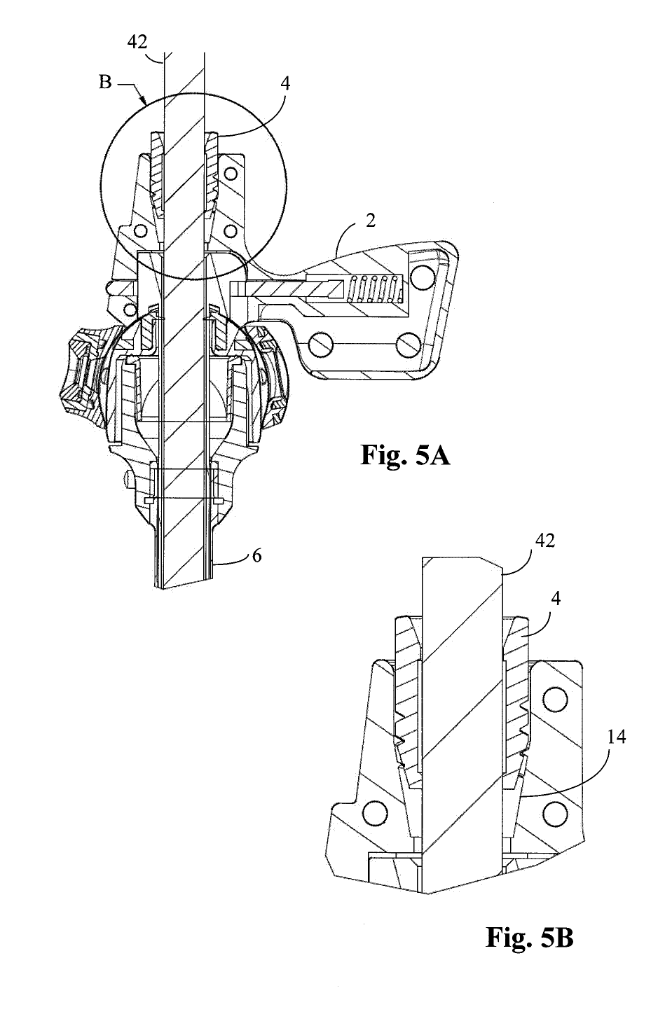

FIG. 5 is a top view of a second embodiment of an optical trocar assembly according to the present invention including an inserted endoscope;

FIG. 5a is a partial cross-section along line A-A in FIG. 5;

FIG. 5b is a close-up of the section marked B in FIG. 5a;

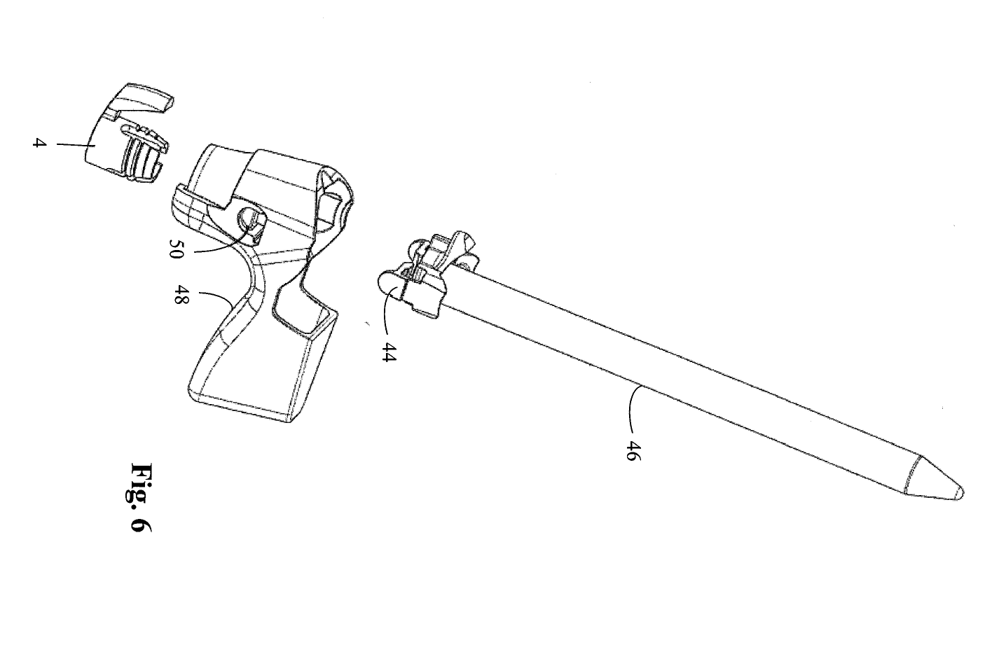

FIG. 6 depicts an exploded diagram of a third embodiment of an optical trocar assembly according to the present invention;

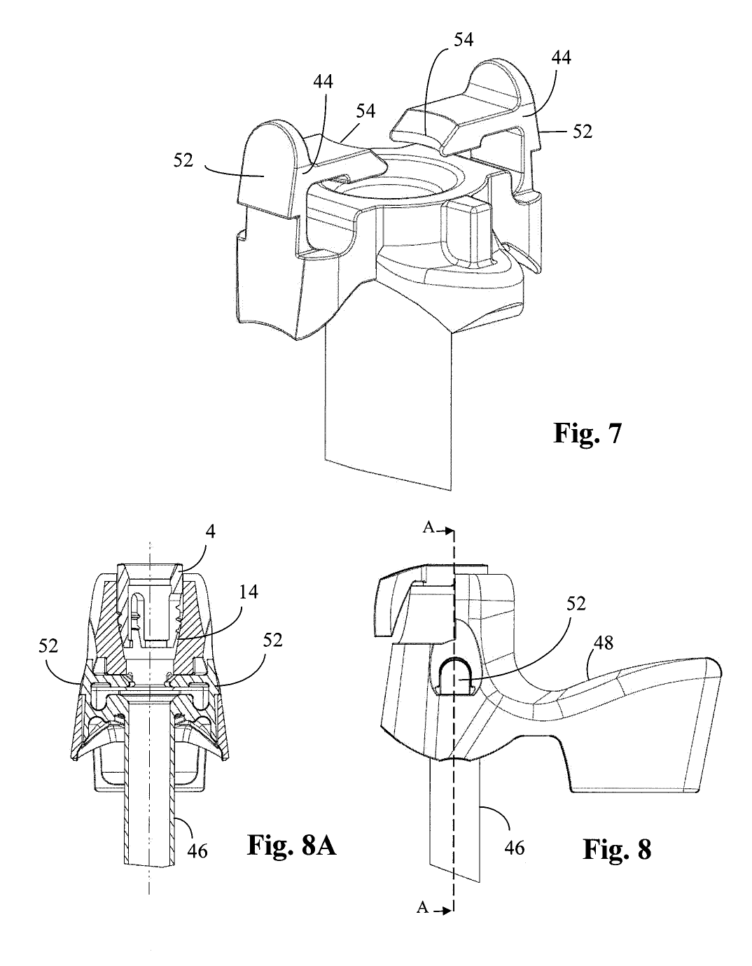

FIG. 7 depicts a close-up of a proximal end of an elongated tubular member used in the assembly of FIG. 6;

FIG. 8 depicts a side view of the optical trocar assembly of FIG. 6 when assembled;

FIG. 8a depicts a cross-section along the line A-A in FIG. 8;

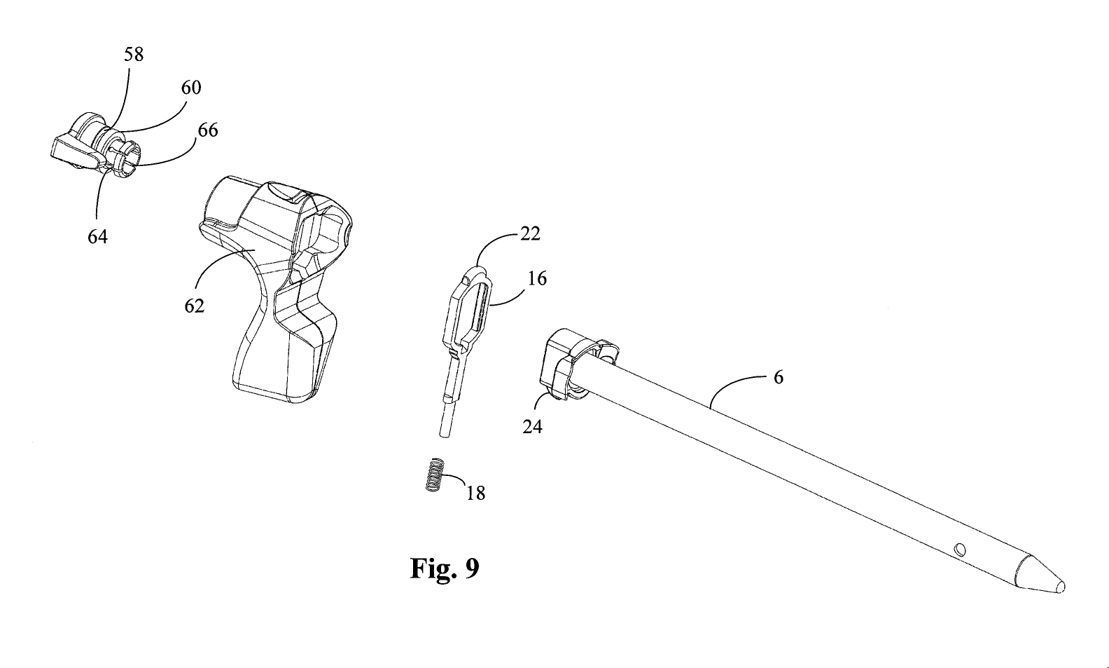

FIG. 9 depicts an exploded view of another embodiment of an optical trocar assembly according to the present invention;

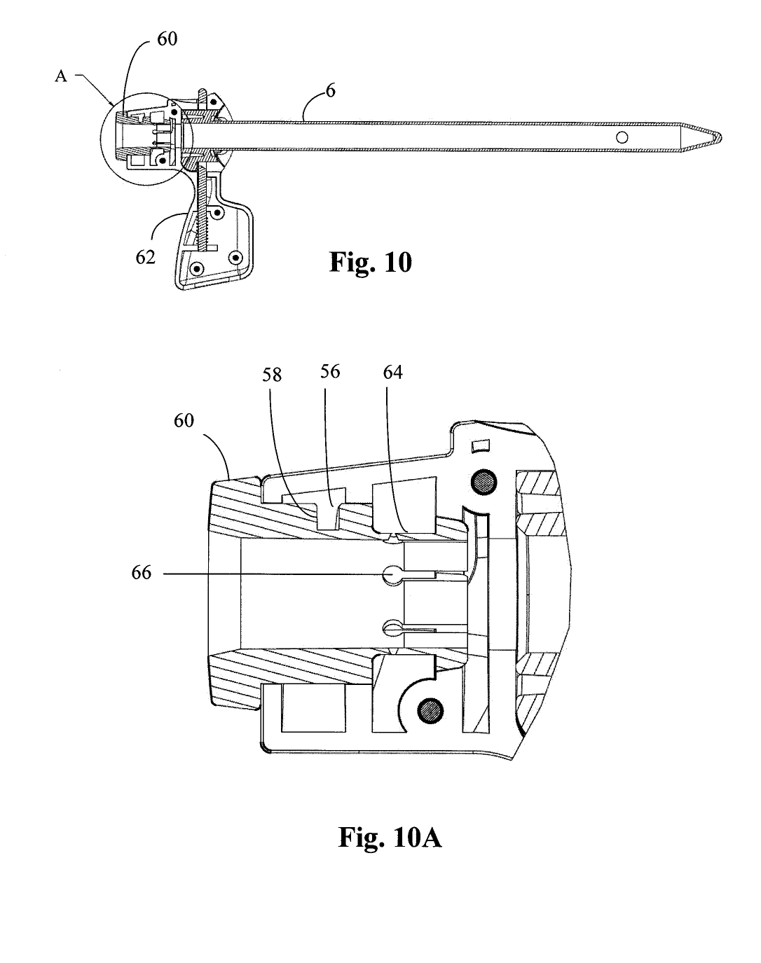

FIG. 10 depicts a cross section of the optical trocar assembly of FIG. 9; and

FIG. 10A depicts a detail view of the section marked A in FIG. 10.

FIG. 1 depicts a side view of an optical trocar assembly comprising a handle 2, a locking collet 4 and an elongated tubular member 6. A top view of the assembly of FIG. 1 is depicted in FIG. 2 and a cross-section in FIG. 2a. An exploded view of the assembly of FIG. 1 is depicted in FIG. 3, with the distal portion of the elongated tubular member not shown for clarity.

Referring to FIG. 3 and the cross-section of FIG. 2a, it can be seen that the elongated tubular member 6 defines a longitudinal axis 8. The elongated tubular member 6 has a distal end 10 which is formed from an optically clear material. In this embodiment, the distal tip 10 has a generally conical shape with a rounded tip. This shape assists in penetration of the optical trocar assembly through tissue. In alternate embodiments, other shapes of distal end 10 may also be used, for example, more complex tapering or twisted shapes.

The proximal end of the elongated tubular member 6 defines a latch surface 12. The latch surface 12 is substantially perpendicular to the longitudinal axis 8 and faces the distal end of the elongated tubular member 6.

The handle 2 defines a passage which is coaxial with the longitudinal axis 8. It comprises a proximal portion which defines a tapering opening 14 for receiving the locking collet 4.

A distal portion of the passage is configured to receive the proximal end of the elongated tubular member 6. A latch member 16 is provided inside the handle 2 in the distal portion of the passage, it is arranged for movement perpendicular to the longitudinal axis 8. A helical spring 18 is also contained within the handle 2, and biases the latch member 16 into a closed position wherein a portion of latch member 16 extends into the distal portion of passage 2. As can be seen in FIG. 2a, this biased, closed position means that an element 20 of the latch member 16 is positioned within the passage so that it engages the latch surface 12 of the proximal end of an inserted elongated tubular member 6. When the latch member 16 is in the closed position it therefore prevents removal of an inserted elongated tubular member 6.

Latch member 16 also comprises a projection 22 which extends outside the handle 2 through an opening in the handle 2. A user may press projection 22 to move latch member 16 perpendicular to longitudinal axis into an open position, against the force of spring 18. In the open position, element 20 no longer engages latch surface 12 so that the elongated tubular member 6 can be removed from the handle 2.

To enable insertion of the proximal end of the elongated tubular member 6 into the handle 2 without requiring the latch member 22 to be manually moved into the open position, the proximal end of the elongated tubular member 6 includes a curved, tapering surface 24 which acts to move the latch member 16 into the open position as the elongated tubular member 6 is inserted proximally into the handle 2. Once the elongated tubular member 6 is fully inserted, the latch surface 12 moves proximally beyond the element 20 of the latch member 16 and the latch member 16 returns to the closed position under the force of the spring 18.

The handle 2 comprises a grip 26 which extends generally perpendicularly relative to the longitudinal axis and which is sized to be held within a user's hand. The latch member 16 is contained within the grip 26 in this embodiment, improving the ergonomics of the resulting assembly.

The locking collet 4 comprises a through bore sized to receive an endoscope in use. It also defines a tapering external surface 28 with a thread 30 provided to engage a corresponding thread 32 formed on the tapering portion of the passage of the handle 2. An enlarged cross-section showing the interaction between the locking collet 4 and the handle 2 is depicted in FIG. 4. The locking collet 4 also includes longitudinally extending slots 34 which define flexible members that move inwardly, towards the longitudinal axis as the collet 4 is advanced longitudinally into the handle 2. This longitudinal advancement occurs by rotation of the locking collet 4 relative to the handle 2, the action of the screw threads 32 and 30 draw the locking collet 4 into the handle 2. The tapering opening 14 and tapering external surface of the locking collet then act as a cam surfaces to force the flexible members defined by the slots 34 inwards, reducing the effective internal diameter and gripping an inserted endoscope.

A rotation member 36 extends radially outward from the collet 4. This rotation member 36 allows a user to easily rotate collet 4 and also provides improved mechanical advantage. The handle 2 includes circumferential stops 38 which engage rotation member 36 to limit the possible rotation of the collet 4 relative to the handle 2. This provides improved ease of use because the locking state of the collet can be immediately determined visually. It also prevents over-tightening of the collet 4 which could damage the optical trocar assembly or an inserted endoscope.

This embodiment therefore provides an optical trocar assembly which has a minimum number of parts and reduced complexity in both assembly and disassembly. Preferably, the handle 2, collet 4 and elongated tubular member 6 are manufactured from plastic materials which can be autoclaved, permitting sterilisation/decontamination of the assembly after use and enabling reuse of the components. Examples of suitable materials include polyoxymethylene (POM), polyphenolsulfone (PPSU) and polyether ether ketone (PEEK). Latch member 22 is preferably also manufactured from a plastic material. The optical trocar assembly may be designed to be disposable after use, in which case it is not necessary for the material to be suitable for autoclaving is not required.

A second embodiment of the present invention is depicted in top view in FIG. 5. This embodiment is the same as the embodiment of FIG. 1, except as described below. This embodiment also includes a port assembly 40. The port assembly 40 provides the working channel for a surgical procedure. For example, if the optical trocar assembly of this embodiment provides the first port assembly in a surgical procedure then the port assembly 40 may be used for insufflation and relieving the endoscope prior to insertion of other ports.

A cross-section through the assembly of FIG. 5 is shown in FIG. 5a. This shows more clearly how the system retains the components of a handle 2, collet 4 and elongated tubular shaft 6 of the first embodiment. The cross-section in FIG. 5a shows an endoscope 42 inserted into the assembly. FIG. 5b shows a close-up of the collet 4 within the tapering opening 14 of the handle 2. It can be seen how the internal diameter of the through bore through the locking collet 4 has been narrowed so that the flexible member of the locking collet 4 engage the endoscope 42 to lock it in place both rotationally and axially relative to the handle 2. This engagement distributes the locking force over an area of the endoscope shaft, spreading the load and reducing the likelihood of damaging the endoscope.

FIG. 6 depicts an exploded view of a third embodiment of the invention. The construction of this embodiment is the same as the embodiment of FIG. 1 except as described below.

In this embodiment the latch member in the handle and the latch surface on the elongated tubular member are omitted. Instead, a pair of diametrically opposed latch members 44 are formed on the proximal end of the elongated tubular member 46. The latch members 44 are integrally formed with the elongated tubular member 46 from a resilient material, preferably a resilient plastics material which is suitable for autoclaving. The latch members 44 comprise a projection 52 which extends in a radial direction away from the longitudinal axis. Each latch member 44 can be moved from the first position depicted in FIG. 6 towards the longitudinal axis, so that the projection 52 moves closer to the longitudinal axis.

The handle 48 comprises receptacles 50 for receiving the projection 52 of the latch member 44. To assemble the optical trocar assembly, the proximal end of the elongated tubular member 46 is inserted into the handle 48. The projection 52 has a tapering surface which is engaged by the handle 48. This causes the latch members 44 to deform inwardly, towards the longitudinal axis into the second position. Once the elongated tubular member 46 is fully in place in the handle 48, the projections 52 align with the receptacles 50 and move outwardly using the natural resilience of the material. This results in a snap-fit connection between the elongated tubular member 46 and the handle 48. The snap-fit connection can be released by manually pressing the projections 52 inwards, to disengage them from the receptacles 50.

The latch members 44 each include a curved end portion 54 which is generally aligned with the central passage through the elongated tubular member 46. These end portions 54 are positioned so that when the latch members 44 move inwardly, towards the longitudinal axis, the end portions 54 extend into the central passage defined by the elongated tubular member 46. Therefore, when an endoscope is in place within the elongated tubular member 46, movement of the projections 52 towards the longitudinal axis is prevented because the end portions 54 will be blocked from movement towards the longitudinal axis by the endoscope shaft. This ensures that the elongated tubular member 46 cannot be accidentally released from the handle 48 when an endoscope is in place.

FIG. 9 depicts an exploded view of another embodiment of the present invention. The construction of this embodiment is the same as for the embodiment of FIG. 1, expect as described below. FIG. 10 depicts a cross section of the embodiment of FIG. 9 and FIG. 10A depicts detail of the portion marked A in FIG. 10.

In this embodiment, rather than a thread, a single helical rib 56 and corresponding helical recess 58 are provided to engage the locking collet 60 with the handle 62. In this embodiment the helical rib 56 is formed in the tapering opening of the handle 62 and the recess 58 is formed in the tapering external surface of the locking collet 60, although in other embodiments these may be reversed (i.e. the recess is provided by the handle and the rib by the locking collet). In some circumstances, a single helical rib 56 gives a stronger construction than the thread used in other embodiments.

As can be seen most clearly with reference to FIG. 10A, the helical rib 56 and helical recess 58 do not need to extend through 360°, they are only required to extend around the angle between the circumferential stops 38.

To improve the flexibility of the distal portion of the locking collet 60 to deform inward and grip the shaft of an inserted endoscope, a circumferential recess 64 and cut outs 66 are provided in the distal portion of the locking collet 60.

Embodiments have been described which provide an optical trocar assembly with a simple construction allowing an inserted endoscope to be locked both axially and rotationally relative to the handle. The construction is simple, reducing complexity of manufacture and assembly and disassembly during use. The optical trocar assembly is also well suited for reuse because a minimum number of parts are required to be sterilised/decontaminated.

In all of the above embodiments, if the optical trocar assembly is reused, it is preferable that the number of times the optical trocar is reused is limited. This is because the optical clarity of the distal portion of the elongated tubular member may degrade with repeated sterilisation/decontamination. It is also possible that the integrally formed latch members 44 of the FIG. 6 embodiment may suffer fatigue and weaken with repeated use. It is therefore preferably that the optical trocar assembly is designed to be reused less than 20, more preferably less than 10, still more preferably less than 5 times.

1. An optical trocar assembly comprising:
an elongated tubular member for receiving an endoscope, defining a longitudinal axis and having an open proximal end and a closed, optically clear distal end, wherein the distal end is adapted for penetrating body tissue;
a handle releasably connected to the elongated tubular member and defining a passage which is coaxial with the longitudinal axis and which has a distal portion and proximal portion, wherein the distal portion is for receiving the open proximal end and proximal portion defines a tapering opening which is tapered in the direction towards the distal portion; and
a locking collet positioned within the tapering opening, having a through bore which is coaxial with the longitudinal axis and a tapering external surface;
wherein one of the tapering opening and the tapering external surface comprises a helical rib and the other of the tapering opening and the tapering external surface defines a helical recess for engaging the helical rib.

2. An optical trocar assembly according to claim 1, wherein the locking collet further comprises an integrally formed and radially extending rotation member for rotating the locking collet relative to the handle.

3. An optical trocar assembly according to claim 2, wherein the handle further comprises first and second circumferential stops for engaging the rotation member and limiting the rotational movement of the locking collet to the handle.

4. An optical trocar assembly according to claim 1, wherein the handle further comprises a latch member for releasably engaging a latch surface formed on the proximal end of the elongated tubular member.

5. An optical trocar assembly according to claim 4, wherein the latch member is movable in a direction perpendicular to the longitudinal axis between open and closed positions; and
wherein in the open position the proximal end of the elongated tubular member can be inserted into or removed from the handle, and in the closed position the latch surface of an inserted proximal end of the elongated tubular member is engaged by the latch member.

6. An optical trocar assembly according to claim 4, wherein the latch member is biased into the closed position by a resilient element.

7. An optical trocar assembly according to claim 4, 5 or 6, wherein a portion of the latch member is accessible outside the handle to move the latch from the closed to the open position, against the force exerted by the resilient element.

8. An optical trocar assembly according to claim 1, wherein the proximal end of the elongated tubular member further comprises a latch member having a projection which extends outwardly from the longitudinal axis;
wherein the latch member is movable between a first position, in which the projection is at a first distance from the longitudinal axis and a second position, in which the projection is at a second distance from the longitudinal axis which is closer than the first distance; and
wherein the handle further defines a receptacle which is engaged by the projection when the latch member is in the first position and disengaged by the projection when the latch member is in the second position.

9. An optical trocar assembly according to claim 8, wherein the latch member is biased into the first position.

10. An optical trocar assembly according to claim 8, wherein the latch member is integrally formed with the elongated tubular member.

11. An optical trocar assembly according to claim 8, wherein the latch member further comprises an end portion positioned closer to the longitudinal axis than the projection;
wherein when the latch member is in the first position, an internal cavity of the elongated tubular member is not obstructed by the end portion and, when the latch member is in the second position at least part of the end portion extends into the internal cavity.

12. An optical trocar assembly according to claim 11, wherein the end portion defines a concave curved surface.

13. An optical trocar assembly according to claim 8, wherein there are at least two latch members spaced around the longitudinal axis at the same axial position.

14. An optical trocar assembly according to claim 1, wherein the handle further comprises a grip extending substantially perpendicular to the longitudinal axis.

15. A system comprising:
an optical trocar according to claim 1; and
an endoscope sized to fit within the elongated tubular member, the passage of the handle and the through bore of the locking collet.
